(19) United States
(12) Patent Application Publication
(10) Pub. No.: US 2011/0203349 A1
(43) Pub. Date: Aug. 25, 2011

(54) Animal Scent Detection Apparatus and System

(76) Inventor: David Reese, Chicago, IL (US)

(21) Appl. No.: 12/860,758

(22) Filed: Aug. 20, 2010

Related U.S. Application Data
(63) Continuation-in-part of application No. 11/891,176, filed on Aug. 9, 2007, now abandoned.

(51) Int. Cl. G01N 33/00

(52) U.S. Cl. 73/23.34

(57) ABSTRACT

A portable scent detection system for moving air from a search area to an animal's nose that can include a hand-held pickup for collecting air from a target area, an air moving unit containing at least one fan, a return hose coupled between the pickup and the air moving unit, and a scent presentation device worn on the animal's head with a one or more presentation ports near the animal's nose. The preferred animal is a dog; however, any animal which can be trained to indicate detection of a target scent can be used. The air moving unit can be double or single. A single unit can contain one fan, while a double unit can contain a second fan. More fans can be added in series or parallel as needed to provide adequate airflow. The pickup device can also optionally contain a fan. The fans can be battery powered using fixed or rechargeable batteries. The battery or batteries can be external or internal to the unit. The air moving unit can be contained in a waist or backpack for ease in use. The scent pickup can also optionally contain a filter to filter out dust or unwanted particles. A system of hoses normally can be run between the pickup device and the air moving unit and between the air moving unit and the scent presentation device. The air moving unit can optionally be located in a widened section of the hoses or hoses.

This is a continuation-in-part of copending application Ser. No. 11/891,176 filed Aug. 9, 2007. Application Ser. No. 11/891,176 is hereby incorporated by reference.

### BACKGROUND

1. Field of the Invention

The present invention relates generally to the field of scent detection and more particularly to a animal scent detection system.

2. Description of the Prior Art

It is well known to use dogs or other animals to search for contraband such as drugs or explosives. This is because animals have noses that are far more sensitive and responsive to a wide variety of materials than humans. Dogs, for example, are know to be able to detect various materials in the parts per billion range. Canines have been used particularly because they can be trained to respond in a predetermined manner when they detect the presence of a target substance.

In the modern world, it is becoming more and more important to be able to detect drugs, explosives and other contraband that produces a detectable scent. This is particularly true at airport security check points, national border entry points and other key locations where people and vehicles need to be checked. It is also important to law enforcement personnel to be able to find hidden contraband in premises or business searches or find explosives of contraband in containers and vehicles.

One problem with simply having a dog sniff for substances in hand carried luggage or at a check point is that such a procedure may annoy or frighten an innocent person, or it might even be an unacceptable invasion of privacy. This is particularly true in a place like an airport security check point where numerous people of all ages and genders routinely pass through. A second problem arises in buildings where target substances may be hidden out of smelling range of an animal walking on a leash (contraband hidden above ceiling tiles for example). This problem might even arise with persons passing through a check point if the substance was hidden on their body high enough to put it above the dog's smelling range.

It is known in the art to use a pass-through portal with air taken from the portal and passed to a dog or a machine. However, this technique is not portable for use in building searches, and it generally requires the dog to stay very near one point in the equipment such as an opening where air samples are directed.

It would be advantageous to have a portable apparatus that would allow security and law enforcement personnel to take advantage of the sensitivity and selectivity of a canine's nose (or any other animal) to detect trace scent from target materials such as explosives, drugs and the like while walking with the dog through the search area.

### SUMMARY OF THE INVENTION

The present invention relates to a portable scent detection system for moving air from a search area to an animal's nose that can include a hand-held pickup for collecting air from a target area, an air moving unit containing at least one fan, a return hose coupled between the pickup and the air moving unit, and a scent presentation device worn on the animal's head with a one or more presentation ports near the animal's nose. The preferred animal is a dog or canine; however, any animal which can be trained to indicate detection of a target scent can be used. The air moving unit can be double or single. A single unit can contain one fan, while a double unit can contain a second fan. More fans can be added in series or parallel as needed to provide adequate airflow. The pickup device can also optionally contain a fan. The fans can be battery powered using fixed or rechargeable batteries. The battery or batteries can be external or internal to the unit. The air moving unit can be contained in a waist or backpack for ease in use. The scent pickup can also optionally contain a filter to filter out dust or unwanted particles. A system of hoses normally can be run between the pickup device and the air moving unit and between the air moving unit and the scent presentation device. The air moving unit can optionally be located in a widened section of the hoses or hoses.

The system can be used by attaching the air moving unit and batteries in a waist or backpack with the scent presentation device strapped to a canine's nose. The operator and animal can then sweep and area using the pickup device to direct scents to the canine. The animal can be trained to appropriately signal when a target scent is detected.

The system can also be used to read out filters collected from rooms, containers, trains, planes, automobiles and other locations. The filters can be read out in real time by simply routing air from the filter to the animal at a convenient time and location.

### DESCRIPTION OF THE DRAWINGS

Several drawings are being provided to illustrate embodiments of the present invention.

FIG. 1 shows a dog being used with a portable embodiment of the present invention to detect scents.

FIG. 2 shows a muzzle-like device that can be worn by a dog or other animal that can direct scents to the animal's nose.

FIG. 3 shows details of a portable embodiment of the present invention.

FIG. 4 shows internal details, including the possible location of fans, of the embodiment of FIG. 3

FIG. 5 shows a different embodiment of a portable unit.

FIG. 6 shows an embodiment of the invention with a fan embedded in an enlarged portion of tubing.

FIG. 7 shows a optional auxiliary fan unit that can be attached to embodiments of the invention for searching larger areas such as containers, truck cargo bays and the like.

FIG. 8 shows a sample concentrating device.

FIG. 9 shows an air ionizer device.

FIG. 10 shows a method of injecting ionized water into a flow.

FIG. 11 shows a remote container sample collector.

FIG. 12 shows electronic airflow monitoring device.

FIG. 13 shows a heated air movement system to raise temperature in a search area.

Several drawings and illustrations have been presented to aid in understanding the present invention. The scope of the present invention is not limited to the embodiments or details shown in the figures.

### DESCRIPTION OF THE INVENTION

The present invention relates generally to an apparatus and system for directing scents to a dog's or other animal's nose so that the natural sensitivity and selectivity of the animal's nose can be used to detect concealed target substances such as drugs or explosives. Generally, a dog has to be specially trained to detect very low levels of such scents.

One of the objects of the present invention is to pick up air from the immediate vicinity of the target substance and move that air to the animal's nose in such a way that the search area can be at any height, and done in a way that the animal can move around freely. The term dog or canine will be used in this application to represent any animal that can be trained and used to respond to scent. While dogs are the most common animals that can be used in this application, and are preferred because of their intelligence and ability to be trained, any other animal that can be trained to respond may be used and will be included under the name canine or dog in the present application.

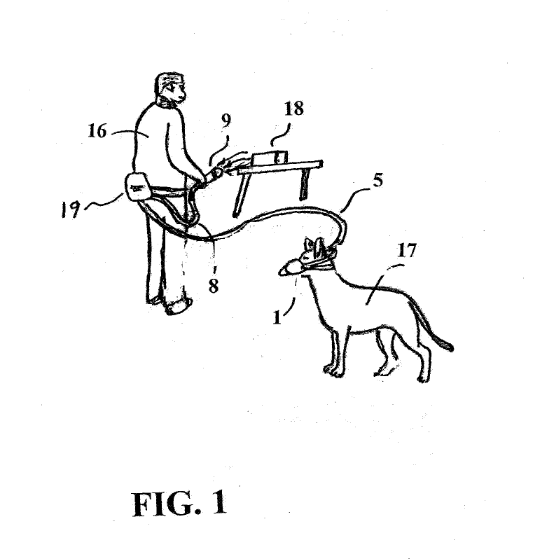

A particular embodiment of the present invention includes a muzzle-like device that fits over the dog's snout and can be loosely and comfortably strapped to its head. Air samples taken from a search area can be directed into a fan unit and through tubing to this device. FIG. 1 shows an example of this embodiment. A dog 17 wearing such a device 1 cooperates with a human operator 16 who wears a portable fan and battery unit 19 in a waist pack. Searching is performed by the operator 16 using a hose 8 and inlet pickup device 9. The small portable unit 19 runs on batteries and moves air from the pickup device 9 to the scent presentation device 1 on the dog's nose via a tube or hose 5. The dog can be trained to respond in a predetermined manner such as sitting upon detection of a target scent. The device can be operated by placing it on the dog, and starting the fans making generally sure that fresh air (with no contamination from any target substance) is entering the pickup. After airflow is established, the operator and dog can proceed to a search area. The portable unit 19 can be worn by the operator during the search. The operator-dog team can then proceed on foot to perform the search for contraband 18. While FIG. 1 shows a item on a table to aid in the understanding of the invention, in reality, contraband 18 may be hidden in walls or ceilings, under beds, in cabinets or anywhere.

A room can be searched quickly using the apparatus of the present invention by moving the pickup to the vicinity of points of interest including the walls or ceiling. The dog can be trained to give an indication of a detection by either moving its body (sitting for example), or by making some other type of indication. Training of this type is unique in itself. Generally when a detection is made, the operator can optionally move away from the locus with the dog, make sure the pickup is pulling in only clean air at that point, wait until the dog has stopped responding, and then return to the suspect area. If the dog gives a second indication of a hit or detection, the operator or other personnel can search for the actual contraband.

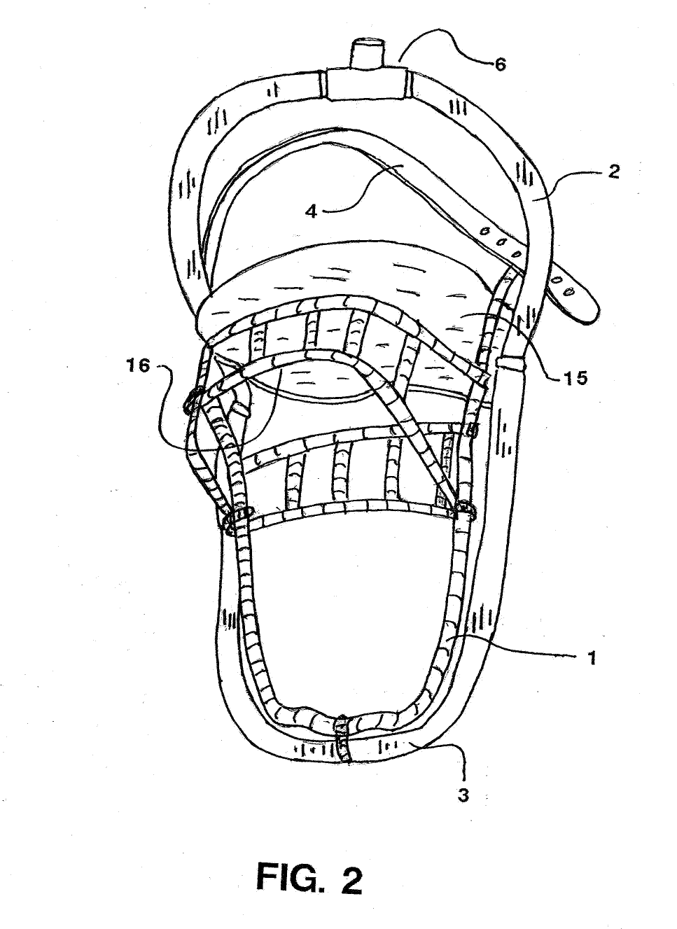

FIG. 2 shows a detail of an embodiment of the muzzle-like scent presentation device that can be worn by the dog. The device can have a rigid metal or plastic frame 1 that is shaped to fit over an animals snout so that the nose is near the front of the device. The device can include an over-bar 16 that passes over the nose. Incoming airflow can enter a T-junction 6 that directs the flow into tubing 2 on each side. While tubing is shown in FIG. 2 on both sides, tubing on only one side is within the scope of the present invention. Holes 3 in the tubing 2 direct the flow to the nose. The holes or other presentation ports or means 3 can be far enough away from the nose so that the dog is comfortable and yet can detect target substances in the desired sensitivity range. The device can be fitted onto the animal's head with a harness 15 that can attach with one or more straps 4. The harness 15 and strap(s) 4 can be made from leather, artificial leather or any other flexible material. The strap can contain holes that mate into a buckle.

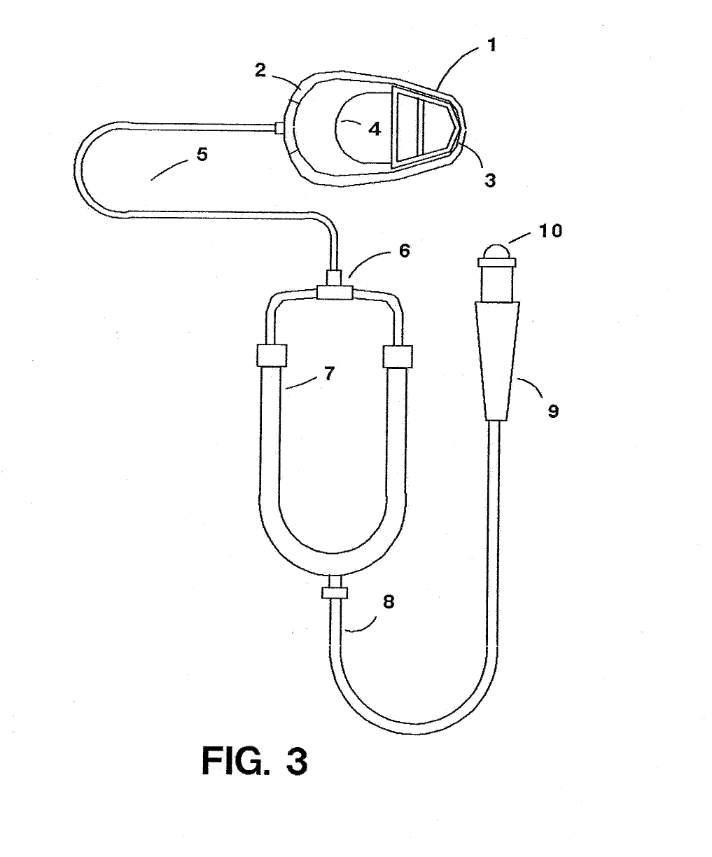

FIG. 3 shows an embodiment of a portable apparatus using the principles of the present invention. A central portable unit 7 can contain fans and provide an air moving point to pull air from an inlet hose 8 and pickup 9. Air can move through the portable unit 7 and be forced out both sides to a junction 6 where it can enter a single feed hose 5. The feed hose 5 can be coupled to the muzzle-like device 1 where air is taken through the one or more feed hoses 2 to a section with exit holes or other presentation ports 3. The muzzle-like device 1 can be held on the animal's head with a harness and one or more straps 4. Air entering the entrance 10 of the pickup 9 can optionally be filtered if desired.

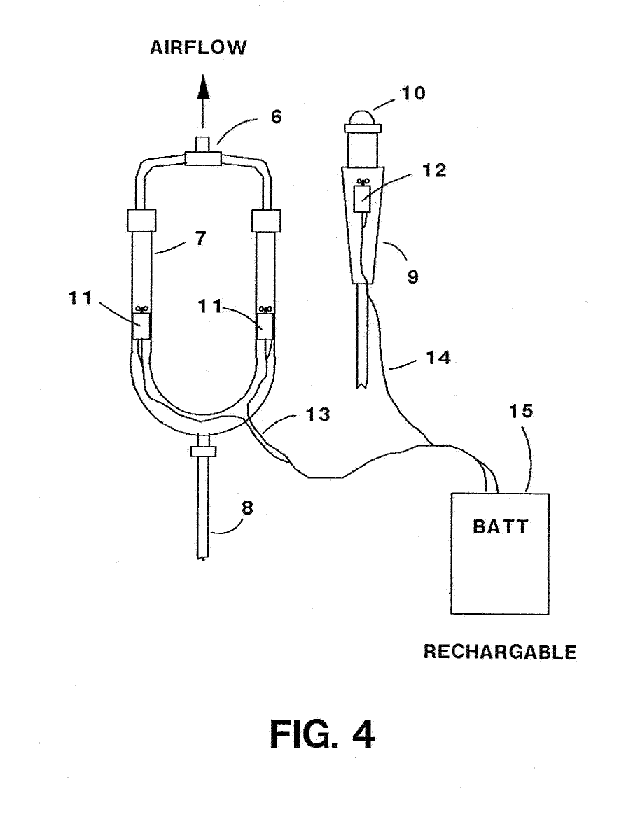

FIG. 4 shows a cut-away detail of the embodiment of FIG. 3. The central portable unit 7 can include one or more internal fans 11. The pickup unit 9 can optionally also contain a fan 12. A preferred fan is a miniature DC fan of model number DB04048B12U made by AVC Company of Taiwan. Any similar fan can be used. The fans can be connected to an electrical source such as a battery 15 by internal or external wires 13, 14 or can be run from conventional power. The battery 15 can optionally be rechargeable.

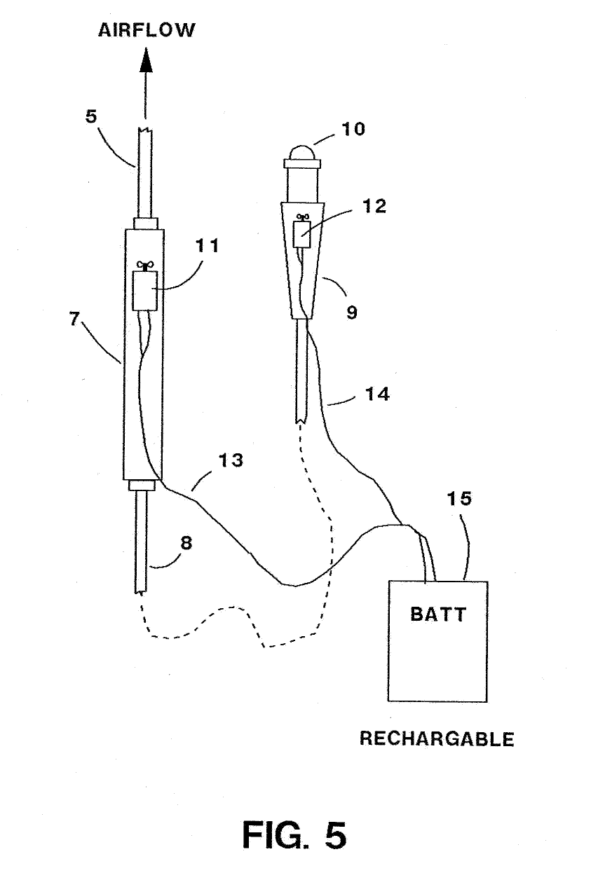

FIG. 5 shows a cut-away view of an alternative embodiment. In this embodiment, the air moving chamber 7 includes a single unit with a single fan 11. The hosing is similar in this embodiment to that of the embodiment shown in FIGS. 3-4 except that no T-junction is needed at the air moving chamber. Again, an optionally rechargeable batter 15 can be used. In addition to using a battery, the unit can be operated from line power such as conventional 115-120 volts (or 220 volts).

In the embodiments of FIGS. 3-5, the battery 15 and the air moving unit 7 can be combined into a pack 19 that can be conveniently worn on the waist or back (shown in FIG. 1). Any other type of pack or of carrying the unit is within the scope of the present invention including straps on the air moving unit 7. In yet another embodiment, the battery can be reduced in size and located inside the air moving unit.

In some cases, the search area may be too large or full of materials, or there may not be time to search it. In this case, larger fans can be used in the area to direct scents to the portable unit or through a larger hose directly to the animal's nose.

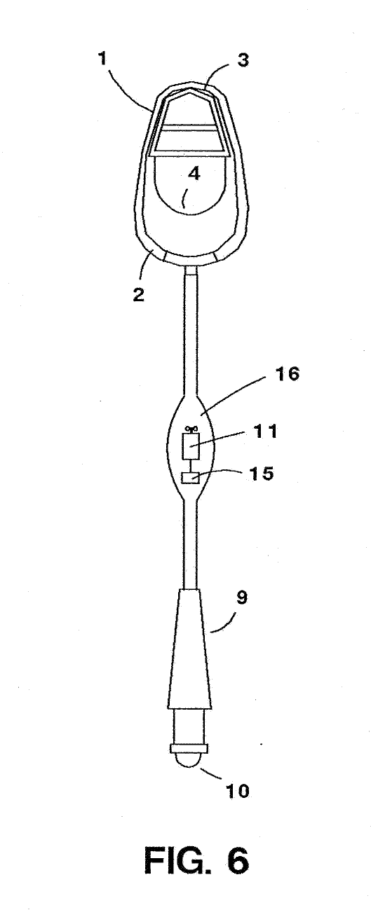

It should be noted in the embodiments of the present invention that the air moving unit can optionally be incorporated directly into an enlarged section of the hose if desired. FIG. 6 shows an embodiment of the invention containing this feature. A widened section of the tubing 7 can contain a miniature fan 11 or a fan 11 and battery 15.

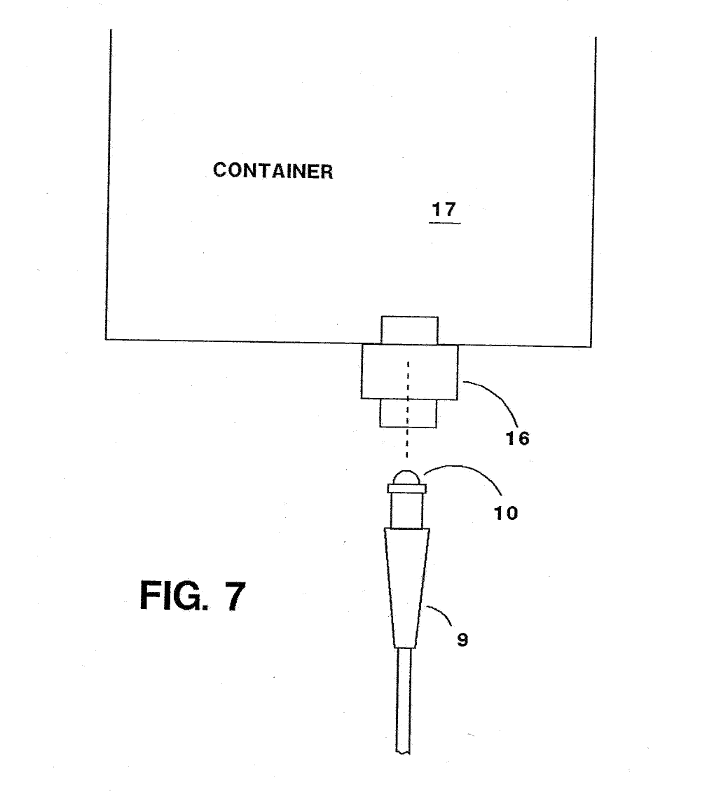

FIG. 7 shows an optional auxiliary external fan unit 16 that has a larger fan or fans that is capable of moving a larger volume of air. This embodiment of the invention is useful for sweeping a large area that is possibly inaccessible such as a cargo container. The larger fan can pull the air out of the container 17 and then directly into the rest of the system as shown in FIG. 7. The external fan unit 16 can be made small enough to carry in the bag 19 (FIG. 1) with the rest of the equipment and plug directly into a fitting in a container or truck. The external fan unit 16 can also be used as a stand-alone unit if desired. The preferred capacity of the external unit is around 400 CFM; however, any capacity is within the scope of the present invention.

It should be noted that the present invention finds many applications beyond the simple searching for contraband. In particular, the invention can be used to sweep for buried land mines, munitions and the like. An operator can use an extension to move the probe 10 out ahead of the dog by a reasonably safe distance. In this way, an operator and dog team can advance slowly in an area with buried mines sweeping ahead of them without risking the safety of the operator or animal. In particular, it can also be used to sample re-circulated air on an aircraft such as after boarding using the craft's cabin air system. In this way, an explosives check could be made on the craft before it leaves the gate.

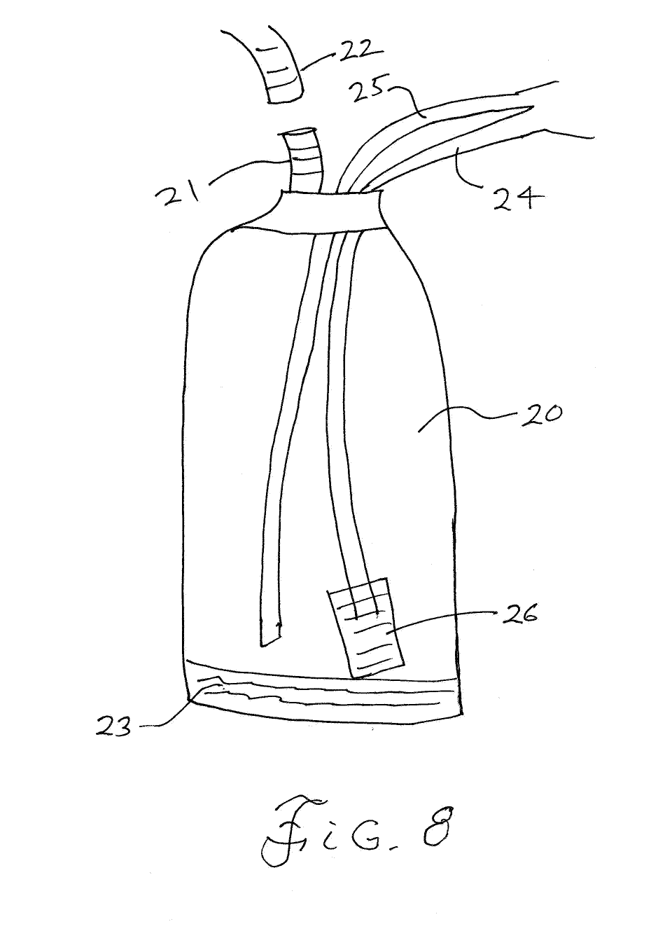

FIG. 8 shows a sample concentration device 20. This device draws air samples in and heats them before delivering them to a dog. Heating the air is desirable since it enhances vaporization of particles. Air is moved a fan in the collection or sniffing device. This air can then be moved into the concentrator 20 via an air inlet port 21. The hose that provides this air 22 can be optionally heated as well. The device 20 can contain a heater 23 in its bottom or elsewhere to heat the incoming air. The air output taken through a filtered port 24, and an optional unfiltered port 25, can be routed to the animal at a lower rate than the air entering the concentrator 20. This second rate can be chosen to be optimum for an animal. A particular rate might be around 118 cubic feet per minute. Any rate is within the scope of the present invention.

The filter 26 could also be a filter removed from a sample site location. Heating the air and forcing it through a filter 26 causes small amounts of a target substance to collect in the filter and, over time, be presented to the animal in higher concentrations. The air collection system with the concentrator can have different adapters depending on the size of the system and the size of the area being searched. The preferred filter 26 is 100% cotton with an inlet flow rate of around 450 cubic feet per minute and an outlet flow rate of around 118 cubic feet per minute. Any filter material and any flow rates are within the scope of the present invention.

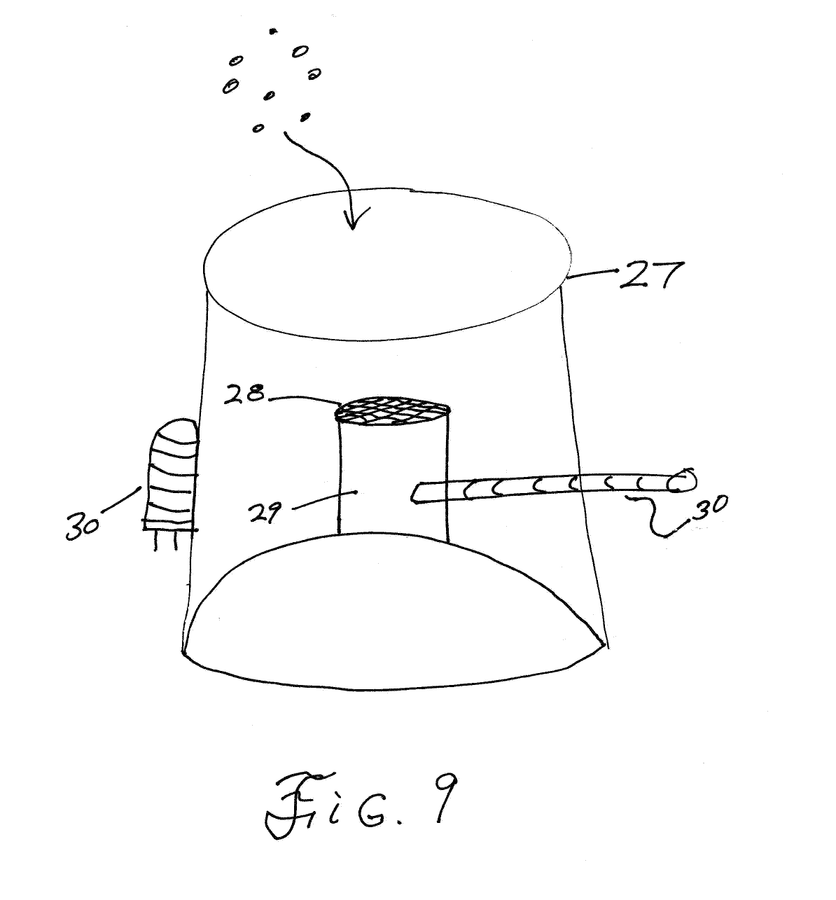

FIG. 9 shows a way of ionizing air being collected so that it more readily collects in a filter. A container 27 holds a filter 28 mounted on a post 29. Air passing through the filter 28 is taken on to the collection machine by an exit hose 29. A cascade ionizer 30 known in the art can be mounted on the container 27 or anywhere near the air entrance to charge surrounding air molecules with a positive electric charge. The filter post 29 is typically metal and is electrically grounded or connected to a negative potential with respect to the positively charged air. The incoming positively charged air is immediately attracted to the negative air filter post 29 and passes through the filter 28. This process causes almost all the charged air to find its way to the filter and thus causes a higher percentage of target molecules to concentrate on the filter 28. The air passing through the filter 28 is subsequently provided to the animal for sensing through the exit hose 29. Volatile vapors and target particles are drawn into the filter 28 and generally collect there. Generally, the electrostatic system (the ionizer) should be turned off before presenting to an animal since the field is generally intense and the animal's hair may stand up. A preferred filter 28 is 100% cotton. Any filtering material and any flow rate is within the scope of the present invention.

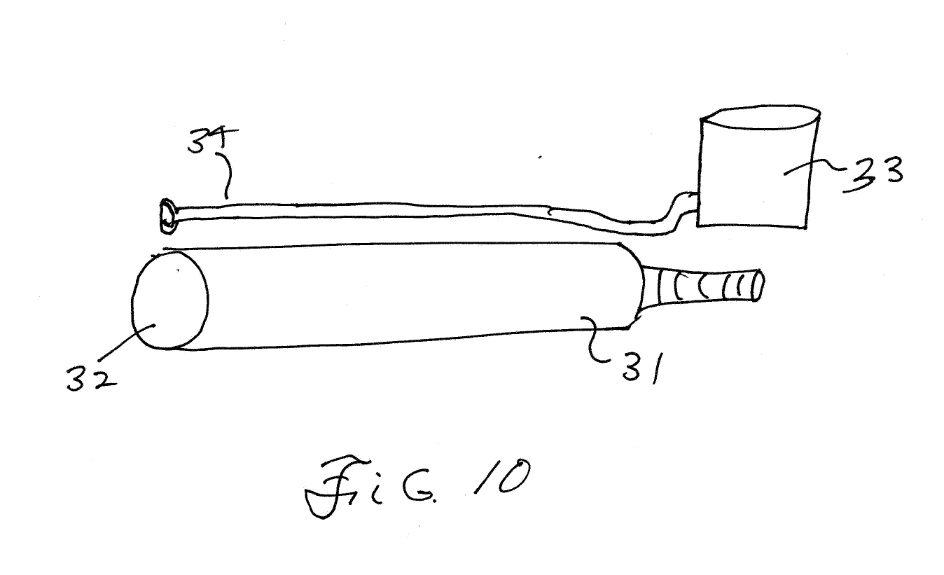

FIG. 10 shows a way of supplying ionized water into the incoming airflow. A flow collector 31 draws air into an inlet port 32. A container 33 holds ionized water and mists it through a mist nozzle 34 an into the incoming air stream. A higher humidity helps an animal identify a target substance. Ionized water is particularly useful since it gives the air a charge which can later in the flow be directed to a filter if desired.

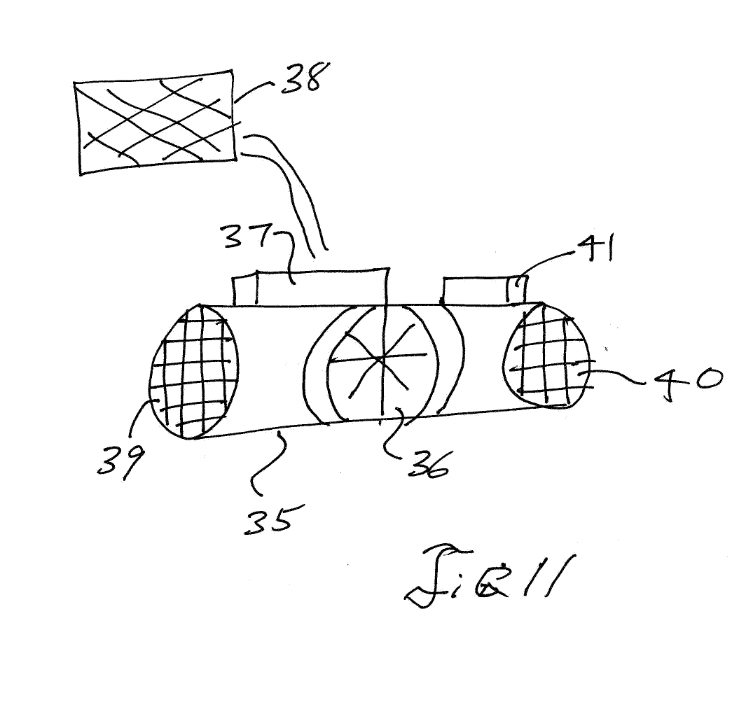

FIG. 11 shows a remote collection unit 35 that can be placed in a container, the hold of a ship, a cargo compartment of an airplane or any other space where target molecules can be collected over time. The device 35 can also be permanently mounted in the base of a pallet for screening warehouses. A fan 36 driven by a battery 37 can run continuously or intermittently. The battery 37 can be a rechargeable battery like a lithium battery or the like known in the art. An optional solar panel 38 can recharge the battery 37 in those locations where sunshine or other light is available. One or more filters can be placed in the stream for collection. An inlet filter 39, an outlet filter 40, or a combination of both an inlet and outlet filter can be used. The device can run on battery circulating air from inside the space through the filter for a period of time. This period may vary depending on the location. For a container, on a ship, the period might be the length of the voyage. For a train or airplane, the period might be trip time, or might be only a few minutes. The period can vary from several minutes to days or months or longer. Normally, the period must have a minimum interval long enough to get sufficient concentration for the animal to detect.

The device can be read out after the particular time period by ether removing the filter(s) 39, 40 and putting them in a sample machine connected to an animal, or by simply running air through the filters in situ in the location and then routing that air to the animal. The particular time period can be variable depending on the location, size of the space an other operational factors. The particular time period can be an interval with a minimum amount of time. The minimum should be chosen to assure a high probability of detection of target molecules by the animal. An optional RF transceiver 41 can be used to remotely control or monitor the device. Any type of wireless technique known in the art can be used with this transceiver including radio of any frequency or modulation and light.

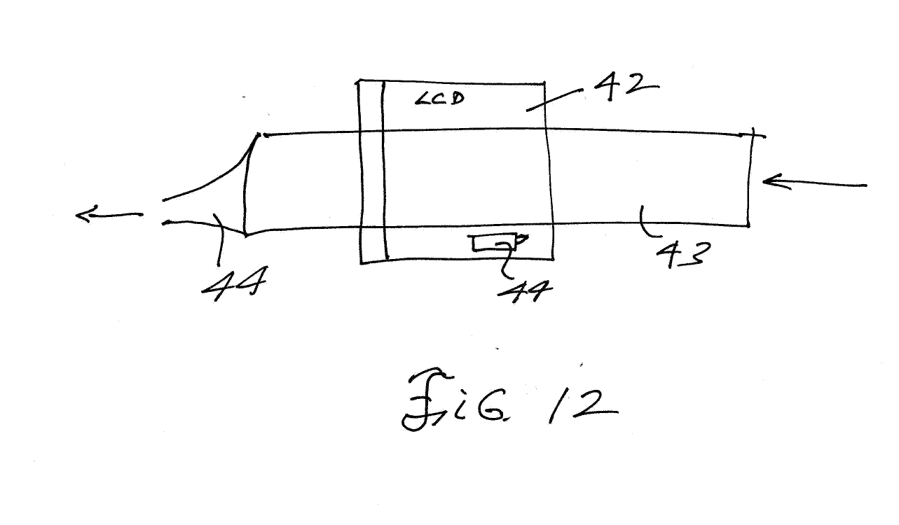

FIG. 12 shows schematically a remote computerized monitoring system especially useful in training dogs to work with the systems of the present invention. The device 42 can maintain a complete training log for one or more animals by recording time, temperature, humidity, flow rates, barometric pressure, battery charge, battery life an any other parameters. The device 42 can be coupled to a flow-through port 43 that takes incoming air samples and moves them out to an outlet port 44 and to various sampling techniques such as a concentrator whose output will be presented to a dog, a direct muzzle, a stainless steel sampler. The device 42 can be powered by a battery 44 that can be rechargeable.

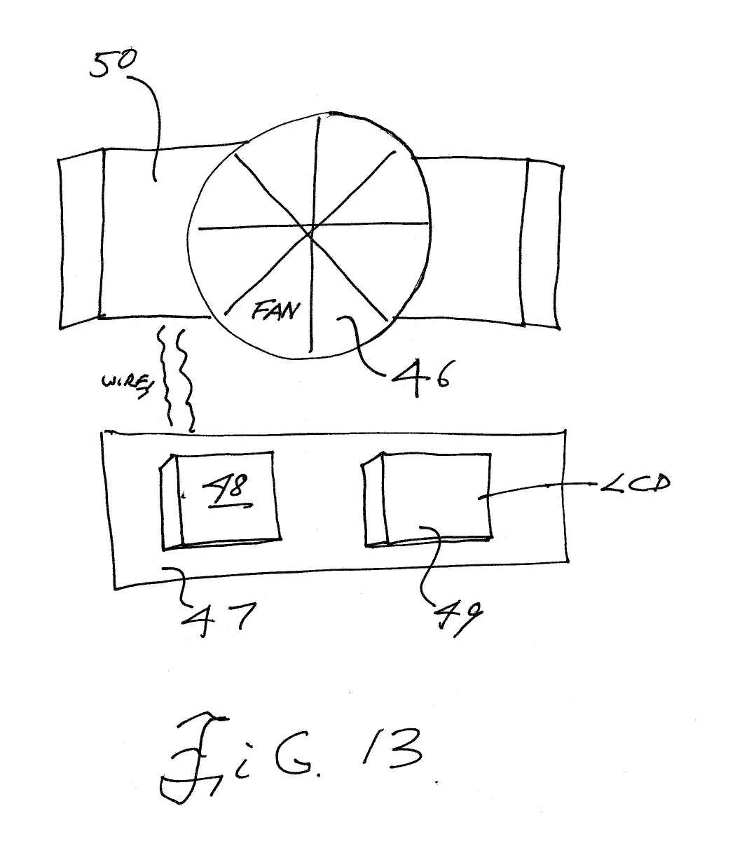

FIG. 13 shows heating unit 45 with a fan 46 and a heating unit 50 that can be placed in a container or other closed space to raise the temperature before samples are taken. It is desirable to raise temperature so that volatile materials increase their rate of vaporization. The unit can be optionally controlled by a wired or wireless local unit 47 in communication with more remote units. The local unit can contain a wireless transceiver 48 and an optional display 49 such as an LCD display. The local unit 47 can optionally monitor flow rate and temperature and can communicate that information wirelessly to a remote location. The fan 46 can be a 12 volt fan or any other type of fan. The heater coil 50 can optionally run at the same voltage or different voltage. Typically, the entire unit would be powered by a battery.

It is optionally also beneficial to pump compressed air into a space before samples are taken. This stirs up the air and is beneficial by lifting particulates and putting them into the air current for sampling and sniffing by the animal.

Embodiments of the present invention can be built into train cars, airplanes, busses and other public transportation. When the train stopped, or before it reached a destination, a dog could read out each car very quickly immediately indicating any problem with a particular car. This would be especially advantageous in a search for explosives aboard commuter trains. The train could be stopped briefly before entering a large metropolitan station (while still safely away), and the entire train, or each car, could be quickly read out by a single trained dog. Shipping containers on ships could contain embodiments of the present invention and be read out either at sea before entering a port, or more likely, just after being unloaded. The read out could be made by simply pumping air through the installed filter unit and feeding this to a single dog. This would allow the entire container to be approved for various contraband such as drugs, illegal humans, explosives or the like without having to open it or try to determine its contents. The present invention can also be used at military or police security checkpoints. Given a line of cars at a checkpoint, each car could be given a small battery-powered collection unit and told to close all windows. When the car reached the checkpoint, the unit could be handed to a guard who could then immediately read its filter using a dog either visible or concealed in a building. The dog would immediately indicate any vehicle with problems.

It should be noted that special training is required before a canine can be effectively utilized with the present invention. Conventional explosive dogs and drug dogs are not effective until they are retrained since they are accustomed to having quite larger concentrations of target materials to work with. The training consists of working the dogs with lower and lower thresholds of target material over an extended time period until they prove themselves reliable and effective at the concentrations typically presented by the present invention. Generally, the training is performed in steps of decreasing concentration where each step may last from days to weeks depending on the particular dog. Such training can coordinated and recorded using a smart phone application or by a program running on any computer.

Several descriptions and illustrations have been provided to better aid in understanding the present invention. A person with skill in the art will realize that numerous changes and variations are possible without departing from the spirit of the invention. Each of these changes and variations is within the scope of the present invention.

I claim:

1. A method of searching for target materials in a confined space comprising:
drawing air into a canister at a first flow rate, said canister containing a heater device;
drawing air out of said canister through a filter at a second flow rate, said second flow rate less than said first flow rate, and wherein said air drawn out has a higher temperature than said air drawn in;
presenting said air drawn out to a canine specially trained to indicate low level concentrations of said target materials.

2. The method of claim 1 wherein said filter is 100% cotton.

3. The method of claim 1 wherein said air drawn in is preheated in an inlet hose.

4. The method of claim 1 further comprising positively ionizing said air drawn in and grounding said filter.

5. The method of claim 1 further comprising misting ionized water into said air drawn in.

6. A method of searching for target materials in a confined space comprising:
placing a canister containing an electric fan and at least one filter element in said confined space;
running said fan from a battery causing air from inside said confined space to circulate through said filter;
leaving said canister, fan and filter with said fan running in said confined space for a time period; after said time period,
causing air to pass though said filter and be presented to a canine specially trained to indicate low level concentrations of particular target molecules.

7. The method of claim 6 further comprising placing an air heating unit in said confined space for a predetermined interval during said time period.

8. The method of claim 6 wherein said is caused to pass through said filter and presented to said canine via a hose or tube between said canister that passes through an outside wall of said confined space.

9. The method of claim 6 wherein said confined space is a container.

10. The method of claim 6 wherein said battery is recharged by a solar panel.

11. The method of claim 6 wherein said confined space is chosen from the group comprising a train, an airplane and a motor vehicle.

12. The method of claim 6 wherein said confined space is a motor vehicle stopped at a security checkpoint.

13. The method of claim 6 wherein said fan runs intermittently.

14. A method of finding target molecules in a confined space comprising;
placing a battery-powered fan and filter in said confined space, wherein said fan causes air from said confined space to pass through said filter for a variable interval of time, said variable interval of time having a predetermined minimum amount of time;
removing said battery-powered fan and filter from said confined space;
causing air to pass through said filter and be presented to a dog trained to signal detection of low levels of said target molecules.

15. The method of claim 14 wherein said battery-powered fan and filter also includes an air ionizer.

16. The method of claim 14 wherein said battery-powered fan and filter also includes an air heater.

17. The method of claim 14 wherein said confined space is chosen from the group comprising a train, an airplane and a motor vehicle.

18. The method of claim 14 wherein said battery-powered fan can be wirelessly remotely controlled.

19. The method of claim 14 wherein said battery-powered fan and filter can be wirelessly remotely monitored.
